(19) United States
(12) Patent Application Publication
(10) Pub. No.: US 2023/0410691 A1
(43) Pub. Date: Dec. 21, 2023

(54) LABEL FOR MATRIX TUBES

(71) Applicant: Gourgen AMBARTSOUMIAN, Laval (CA)

(72) Inventor: Gourgen AMBARTSOUMIAN, Laval (CA)

(21) Appl. No.: 18/249,085

(22) PCT Filed: Oct. 15, 2021
(86) PCT No.: PCT/CA2021/051452

§ 371 Date: Apr. 14, 2023

Related U.S. Application Data
(60) Provisional application No. 63/092,146, filed on Oct. 15, 2020.

(51) Int. Cl. G09F 3/02; G09F 3/00; G09F 3/10; G16H 10/40

(52) U.S. Cl. CPC G09F 3/0288; G09F 3/0297; G09F 3/10; G09F 2003/0273; G09F 2003/0229; G09F 2003/023; G09F 2003/0266; G16H 10/40

(57) ABSTRACT

A label for matrix tube may have a facestock configured to have data on a first surface thereof. An adhesive layer may be on a second surface of the facestock. The facestock may define an end label portion, a main label portion and a neck portion between the end label portion and the main label portion, the neck portion being narrower than the end label portion and the main label portion. A method for applying a label onto a matrix tube may also be provided.

### CROSS-REFERENCE TO RELATED APPLICATION

The present application claims the priority of U.S. Patent Application No. 63/092,146, filed on Oct. 15, 2020, and incorporated herein by reference.

### TECHNICAL FIELD

The present application relates to labels for use with matrix tubes, also known as matrix vials, sample tubes, sample vials.

### BACKGROUND OF THE INVENTION

Sample tubes, also known as matrix tubes, are commonly used to manipulate multiple samples in a compact manner, for instance manually or robotically in laboratories. The sample tubes are relatively small in size, and are fitted in racks having multiple wells (e.g., 24-well, 48-well, 96-well). For instance, the matrix tubes come in sizes of 0.3 ml, 0.4 ml, 0.5 ml, 0.75 ml, 0.8 ml, 1.0 ml and possibly others. They are cylindrical in shape, with a diameter usually in the range of millimeters.

As traceability of samples is a necessity, the matrix tube itself is laser-etched or printed with a 2D barcode on its bottom. The laser-etching/printing has an impact on the cost of such tubes.

### SUMMARY OF THE INVENTION

It is an aim of the present disclosure to provide a novel label for matrix tubes.

Therefore, in accordance with an embodiment of the present disclosure, there is provided a label for matrix tube comprising: a facestock configured to have data on a first surface thereof, an adhesive layer on a second surface of the facestock, and wherein the facestock defines an end label portion, a main label portion and a neck portion between the end label portion and the main label portion, the neck portion being narrower than the end label portion and the main label portion.

Further in accordance with the first aspect, for example, the end label portion has a generally circular shape.

Still further in accordance with the first aspect, for example, the end label portion has a diameter of at most 6.5 mm.

Still further in accordance with the first aspect, for example, the neck portion is narrower than the end label portion by having a minimum width being between 10% to 80% of a width of the end label portion.

Still further in accordance with the first aspect, for example, the neck portion has a constant width from the end label portion to the main label portion.

Still further in accordance with the first aspect, for example, the neck portion flares from the end label portion to the main label portion.

Still further in accordance with the first aspect, for example, the main label portion has a generally rectangular or square shape.

Still further in accordance with the first aspect, for example, corners of the main label portion in the generally rectangular shape are rounded.

Still further in accordance with the first aspect, for example, the main label portion has a maximum width a ranging from 3.0 mm to 40.0 mm.

Still further in accordance with the first aspect, for example, the main label portion is wider than the end label portion.

Still further in accordance with the first aspect, for example, the end label portion, the neck portion and the main label portion extend in a lengthwise of the label, the end label portion having a length ranging between 3.0 to 6.5 mm.

Still further in accordance with the first aspect, for example, the neck portion has a length ranging between 1.0 mm and 15.0 mm.

Still further in accordance with the first aspect, for example, the main label portion has a length of at least 5.0 mm.

Still further in accordance with the first aspect, for example, the main label portion has a tear line transverse to a length thereof.

Still further in accordance with the first aspect, for example, the end label portion and/or the main label portion has data or ink thereon.

Still further in accordance with the first aspect, for example, data on the end label portion differs from data on the main label portion.

Still further in accordance with the first aspect, for example, the data on the end label portion is a QR code or 2D barcode, and wherein the data on the main label portion is a barcode.

Still further in accordance with the first aspect, for example, a wireless communication tag or related component may be in the label.

Still further in accordance with the first aspect, for example, a support liner may be provided, the adhesive layer being between the facestock and the support liner, for releasable connection of the facestock to the support liner.

Still further in accordance with the first aspect, for example, the support liner is in adhered to another support liner by another layer of adhesive in a piggy-back configuration.

Still further in accordance with the first aspect, for example, the support liner has a first liner portion and a second liner portion separated by at least one slit.

Still further in accordance with the first aspect, for example, the at least one slit is opposite the main label portion.

In accordance with a second aspect, there is provided an assembly comprising: at least two of the label as described above; and a single one of the support liner.

Further in accordance with the second aspect, for example, the support liner has a first liner portion for each of the at least two labels, and a single one of the second liner portion shared by each of the at least two labels.

Still further in accordance with the second aspect, for example, tear lines are in the second liner portion between adjacent pairs of the at least two labels.

Still further in accordance with the second aspect, for example, the second support liner is in a roll.

Still further in accordance with the second aspect, for example, label position indicators are between adjacent pairs of the at least two labels.

In accordance with a third aspect, there is provided a kit comprising: at least one of the label as described above, and a matrix tube.

In accordance with a fourth aspect, there is provided a method for applying a label onto a matrix tube, comprising: pressing an end label portion of a label against a bottom face of a matrix tube; deforming the label at neck portion to move a main label portion toward a side surface of the matrix tube; and pressing the main label portion of the label against the side surface of the matrix tube.

Further in accordance with the fourth aspect, for example, there may be performed removing a first liner portion from the end label portion and keeping a second liner portion on the main label portion prior to pressing the end label portion against the bottom face of the matrix tube, and removing the second liner portion from the main label portion prior to pressing the main label portion against the side surface of the matrix tube.

Still further in accordance with the fourth aspect, for example, there may be performed inscribing or printing data on the label.

Still further in accordance with the fourth aspect, for example, there may be included scanning a barcode on the label.

Still further in accordance with the fourth aspect, for example, there may be included retrieving an identity of the matrix tube or its content using the scanning of the barcode.

Still further in accordance with the fourth aspect, for example, there may be included storing data or an identity of the matrix tube or its content using the scanning of the barcode.

### BRIEF DESCRIPTION OF THE FIGURES

FIG. 1 is a perspective view of a label for matrix tube as secured to a matrix tube;

FIG. 2A is a face view of the label of FIG. 1 in accordance with a first variant;

FIG. 2B is a face view of the label of FIG. 1 in accordance with a second variant;

FIG. 2C is a face view of the label of FIG. 1 in accordance with a third variant;

FIG. 2D is a face view of the label of FIG. 1 in accordance with a fourth variant;

FIG. 3A is a schematic sectional view of an exemplary construction of the label of FIG. 1, in accordance with an embodiment of the present disclosure;

FIG. 3B is a schematic sectional view of an exemplary piggy-back construction of the label of FIG. 1, in accordance with an embodiment of the present disclosure;

FIG. 4 is a perspective view of a series of the label of FIG. 1, in a roll in accordance with an embodiment of the present disclosure;

FIG. 5 is a perspective view of a series of the label of FIG. 1, in a roll with a two-part support liner, in accordance with another embodiment of the present disclosure;

FIG. 6A is a rear end view of one of the labels of FIG. 5, with a removable liner portion in accordance with another embodiment of the present disclosure;

FIG. 6B is a rear end view of one of the labels of FIG. 5, with another possible removable liner portion in accordance with another embodiment of the present disclosure; and

FIG. 7 is a perspective view of a series of the label of FIG. 1, in a roll with a two-part support liner, in accordance with another embodiment of the present disclosure.

### BRIEF DESCRIPTION OF THE EMBODIMENTS

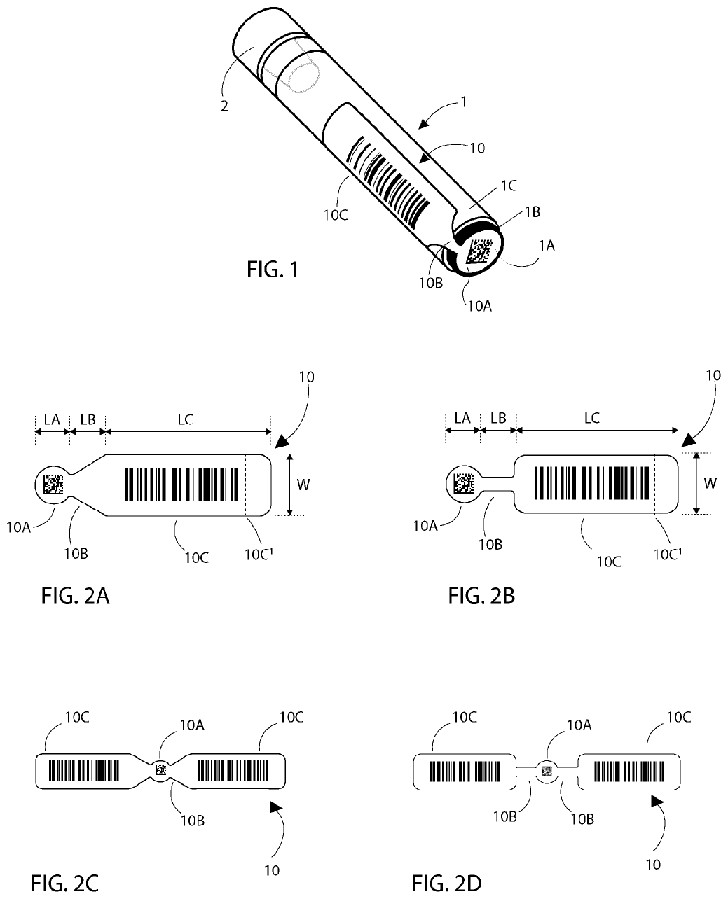

Referring to the drawings and more particularly to FIG. 1, a matrix tube is generally shown at 1. The matrix tube 1 may also be known as matrix vial, sample tube, sample vial. For simplicity, reference is made herein to matrix tube 1, although the tube 1 may be known as other names. The matrix tube 1 may come in different sizes, such as 0.3 ml, 0.4 ml, 0.5 ml, 0.75 ml, 0.8 ml, 1.0 ml, 1.4 ml and possibly others. The matrix tube 1 has a cylindrical shape (as a possibility among others), and is open ended so as to receive a sample(s) in its inner cavity. The matrix tube 1 has a bottom face 1A, and may have a shoulder 1B between the bottom face 1A and a cylindrical side surface 1C, or like side surface if not cylindrical. In the illustrated embodiment, the bottom face 1A is round (as a possible shape among others), and has a diameter of around 5.0 mm, though the diameter of the bottom face 1A may be between 4.5 mm and 6.5 mm, and though other diameters or cross-sectional dimensions outside this range are contemplated. A height of the matrix tube 1 may depend on the volume of the tube 1, and is usually between 20 mm and 49 mm without cap, though other heights outside this range are contemplated. In an embodiment, a central axis of the matrix tube 1 is coincident with a center of the cylindrical side surface 1C.

A cap 2 may be sealingly received in the top open end of the tube 1. In an embodiment, it can be said that the cap 2 is an integral part of the tube 1 (i.e., when referring to “tube 1”, this may include the cap 2), but the cap 2 may not be part of the tube 1. The cap 2 is typically made of an elastomer by which the cap 2 is sealingly received and held captive while capping off the tube 1, for the sample in the tube 1 to be isolated from ambient by the cap 2. The cap 2 may also be a screw cap, for threaded engagement with the tube 1, with appropriate threading being present in the tube 1 and on the cap 2. Other cap materials may be used, the elastomer being an example among others. The caps might have some other parts associated with it such as a swab or brush linked to the internal portion of the cap for the purpose of swabbing a sample from a patient.

Referring concurrently to FIGS. 1, and 2A and 2B, a label 10 for matrix tube 1 is shown as applied to the matrix tube 1 (FIG. 1), or on its own in FIGS. 2A and 2B. The variant of the label 10 of FIG. 2A is shown on the matrix tube 1 of FIG. 1, but the variant of the label 10 of FIG. 2B could also be used in the same manner. The label 10 may have a monolithic or monoblock construction, but may have different parts, such as an end label portion 10A, a neck 10B, and a main label portion 10C. As a variant, as shown in FIGS. 2C and 2D, the label 10 may have a second neck 10B, and a second main label portion 10C extending from the same end label portion 10A, but on the opposite side.

The end label portion 10A is configured to be adhered to the bottom face 1A of the matrix tube 1. In an embodiment, the end label portion 10A emulates the shape of the bottom face 1A, and may be said to be circular, though it may be more appropriate to qualify the end label portion 10A as being quasi-circular as it is tied to the neck 10B and therefore does not describe a full 360-degree circle. The end label portion 10A may have other shapes, including square, squircle, polygon, a combination of shapes, etc. In an embodiment, the end label portion 10A has a diameter ranging from 2.0 mm to 6.5 mm, this range applying to the largest cross-sectional dimension if the end label portion 10A is not circular or quasi-circular. The diameter of the end label portion 10A may be smaller, equal or larger than that of the bottom face 1A. In an embodiment, any type of data may be provided on the end label portion 10A, such as symbols, letters and/or numbers. The data may be pre-printed, or may be printed in situ. The printing of the label 10 may be done after the tube 1 has been labelled, by using for example a tube printing device. In FIGS. 2A and 2B, the end label portion 10A is shown as having the data in the form of a 2D barcode, 3D barcode, or QR code. The data on the end label portion 10A may differ from the data on the main label portion 10C, notably because of size restrictions. For example, the data on the end label portion 10A may be a QR code, 2D barcode, 3D barcode while the data on the main label portion 10C may be a barcode. Other data pairings are possible, including on the neck portion 10B. Furthermore, any portion of the label 10 may have printing such as a logo, trade name, or any image or graphic alone or together with data or information or any indicia.

The neck 10B joins the end label portion 10A to the main label portion 10C. In an embodiment, the neck 10B flares from the end label portion 10A to the main label portion 10C, as in FIG. 2A, in a straight flare as shown or in an arcuate flare. The neck 10B may also be straight, as in FIG. 2B. At the junction with the end label portion 10A, the neck 10B has a width smaller than the diameter of the end label portion 10A (or maximum cross-sectional dimension). This width at the junction may be the smallest width of the neck 10B, and of the label 10 altogether. In an embodiment, the width of the neck 10B at the junction with the end label portion 10A may be between 1.0 mm and 5.0 mm. In an embodiment, the width of the neck 10B at the junction with the end label portion 10A is from 10% to 80% of the diameter of the end label portion 10A (or maximum cross-sectional dimension, or width of the end label portion 10A, i.e., in direction W). In the illustrated embodiment the edges of the neck 10B may be straight, from the end label portion 10A to the main label portion 10C, as in the variant of FIG. 2B. Other shapes are considered, such as arcuate, with a non-linear increasing slope from the end label portion 10A to the main label portion 10C. The non-linear increasing slope may be exponential in shape, from the end label portion 10A to the main label portion 10C.

The main label portion 10C may be generally rectangular or square, or have such an elongated shape (e.g., oval). The main label portion 10C may have a conical shape (i.e., frusto-conical) meaning the free end is wider than the connected end (i.e., at the junction 10C/10B) in order to conform to vials that may have a slightly conical shape. The main label portion 10C may have rounded corners as shown, but the corners may be square, etc. The main label portion 10C is configured to adhere to the cylindrical side surface 1C. The elongated shape of the main label portion 10C is well suited to be adhered to the cylindrical side surface 1C, with the elongated dimension being generally aligned with the central axis of the tube 1. This may reduce the risk of folds when the main label portion 10C is applied to the cylindrical side surface 1C. In an embodiment, the main label portion 10C has a width W (a.k.a., maximum width) ranging from 3.0 mm to 40.0 mm. In an embodiment, the width W is greater than the diameter of the end label portion 10A, or largest cross-sectional dimension if the end label portion 10A is not circular or quasi-circular. While the embodiments show that a an axis of symmetry may be present for the outer shape of the label 10 (horizontal on the drawing sheets), there may be no such symmetry. In an embodiment, any type of data may be provided on the main label portion 10C, such as barcode(s), symbols, letters and/or numbers, or any combination thereof. The label may be opaque, transparent, translucent, hazy or have any degree of opacity, transparency, translucency or haziness or any combination thereof. The label may have a background color, a thermochromic ink, any coating, varnish or lamination. In FIG. 2A, the main label portion 10C is shown as having the data in the form of a barcode, as an example among others. At a junction between the neck 10B and the main label portion 10C, the edges of the label 10 may form obtuse angles, the obtuse angles being in the facestock of the label 10. In an embodiment, the obtuse angles are between 110 degrees and 160 degrees. In the variant of FIG. 2B, the neck 10B may be a narrow strip at a 90 degree angle (or a 90 degree rounded angle—with a fillet as shown) to the label portions 10A and 10C.

By having a portion narrower than the end label portion 10A and than the main label portion 10C, the neck 10B may exhibit a greater flexibility than the end label portion 10A and main label portion 10C. Stated differently, the neck 10B may offer a lesser resistance to deformation, and/or may produce less biasing force when deformed in the manner shown in FIG. 1. The neck 10B in use is indeed curved, and may be in contact with the tube 1 or spaced from it.

In terms of global length of the label 10, as shown in FIGS. 2A and 2B, it may be a combination of dimensions LA, LB and LC, respectively for the end label portion 10A, the neck 10B, and the main label portion 10C. LA may be between 3.0 to 6.5 mm (but could be more, or less), LB may be between 1.0 mm and 15.0 mm, and LC may be between 5.0 mm and 60.0 mm. These values are given as an example. In some instances, the length of the main label portion 10C may be longer such as when there is a need to pass the label 10 over the cap 2 for creating a tamper-evident seal against unauthorized opening of the matrix tube 1. The main label portion 10C would have its end adhered to the cap 2, and could for instance have a tear line 10C′ (a.k.a., tear strip) to tear when the cap 2 is opened. The tear line 10C′ may be a series of perforations in the main label portion 10C or like weakening of the facestock, to enable the manual tearing of the main label portion 10C. The tear line 10C′ may be at other locations. In a variant featuring the tear line 10C′, the main label portion 10C may be used to secure the cap 2. The end of the main label portion 10C extending beyond the tear line 10C′ may have a different shape than a remainder of the main label portion 10C of the label 10 to conform to the cap configuration and size, and facilitate the adhesion of the main label portion 10C to the cap 2. The different shape may start before the tear liner 10C′, such as by a narrowing. The tear liner 10C′ may be in any of the embodiments of the label 10 described herein.

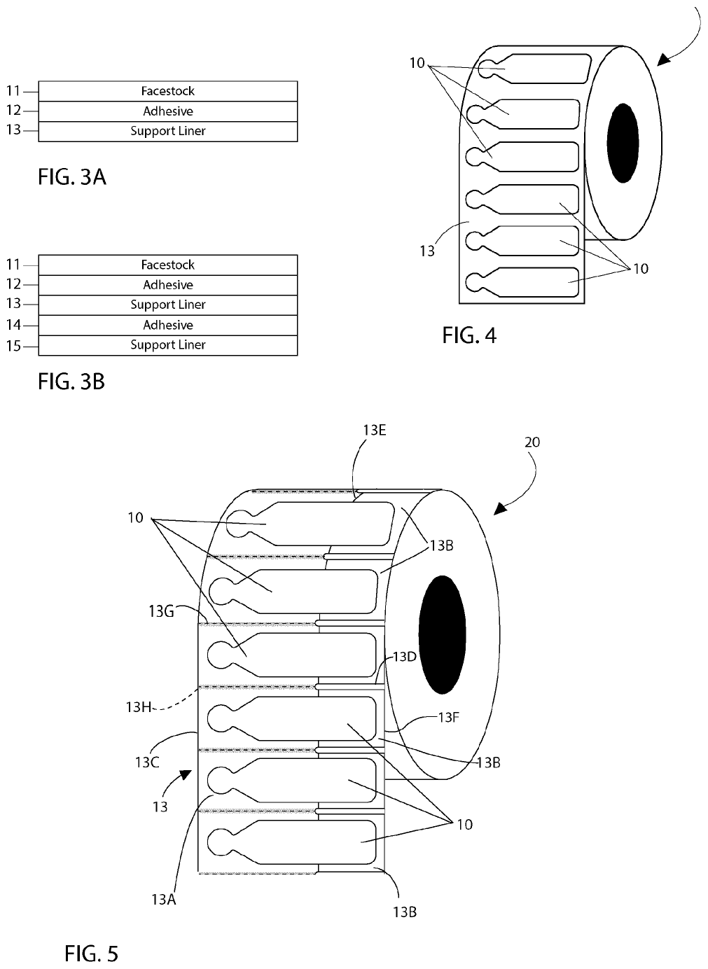

Referring to FIG. 3A, the label 10 may have various layers, all of which may extend from end to end of the label 10, i.e., along a lengthwise direction projecting out of the page of FIG. 3A. Facestock 11 is the surface that can accept ink or printing, although the capability of accepting ink is not a requirement. This may include printing using a thermal printer, a thermal-transfer printer, a direct-thermal printer, a laser printer, an inkjet printer, LED printer, UV printer, impact printer, dot-matrix printer, laser-etching printer, flexographic printer, offset printer or a printing press or any other type of printer or device capable of delivering ink on any of the surfaces of the label. Accordingly, the facestock 11 may incorporate a thermochromic ink system (such as leuco dye), for example in a layer of coating that is part of the facestock or incorporated in the material of the facestock, for the color to be revealed upon heating. The facestock 11 itself may include one or more layers, including transparent and opaque layers, shielding layer(s), top coatings, inks, varnishes, laminations, wireless communication components such as RFID (Radio Frequency Identification), NFC (Near Field Communication), Bluetooth® and other types of wireless communication tags or a chip, sensor, electronic component, etc. The labels 10 can be provided blank or pre-printed, and/or may be with any background color or color indicator such as thermochromic ink, and/or image, and/or information and/or barcode and/or alphanumeric markings, and/or indicia etc. The facestock materials can be made of a polymer, or of a paper such as an impregnated paper. The facestock material(s) may include but is not limited to polymer(s) such as any type of thermoplastic film, any type of polypropylene, biaxially oriented polypropylene (BOPP), polyester (PET), polystyrene, vinyl including polyvinyl chloride (PVC) and polyvinyl fluoride (PVF), acrylate, nylon, satin, polyethylene, polyimide, tyvek, composite materials, silicone, silicone rubber, rubber, synthetic rubber, latex, thermoset plastics, plastic extrusion materials, and may also include cloth tissues, woven or non-woven fabrics, foam, metallic foils such as aluminum foil, Velcro®, degradable and soluble materials including biodegradable materials. The facestock materials include paper and polymer composites or combinations, cloth and polymer composites or combinations, including layers of polymer and paper and/or cloth, such that the paper or cloth is for example laminated with plastic. Embossed, stamped foil, clear, opaque, transparent, translucent and/or reflective materials or any combination thereof may also be used for the facestock 11. The polymers may be thermoplastics or thermos-softening plastics, i.e., plastic polymers that soften or become moldable when heated to a certain threshold temperatures, to solidify upon cooling. The polymers may be thermoset plastics comprising cross-linked polymers to form an irreversible chemical bond and may become non-moldable when heated. The facestock 11 may be selected to have direct thermal printing capacity meaning that an ink system (e.g. leuco-dye) is incorporated inside the facestock 11 or in its top coating. The ink may then be released upon heating the facestock layer 11 such as by a thermal printer or a heat-emitting or heated instrument. The facestock 11 may be for example printed or coated with a non-reversible (e.g., leuco dye) or a reversible thermochromic ink system which may also be also incorporated inside or applied on the facestock or other constituent of the label 10 such as onto the lamination or inside the adhesive. The use of a combination of printing methods is contemplated. All labels 10 of the present disclosure may or may not accept writing by a pen, pencil, marker or any other instrument for inscribing or manual writing.

In some variants, a thickness of the facestock 11 may be equal to or less than about 8.0 mil. More specifically, the label facestock 11 may have a thickness of between about 0.3 mil and 6.0 mil, and even more specifically, the facestock 11 may have a thickness of between about 0.4 mil and 4.8 mil, or of between about 0.8 mil and 3.5 mil, or still more specifically, of between about 0.9 mil and 3.2 mil.

An adhesive layer 12 may be coated on the underside of the facestock 11. A support liner or release liner 13 may also be provided upon which the labels 10 are laid, from which the adhesive layer 12 with the facestock 11 can be released. In an embodiment, the label 10 does not include the release liner 13, though it may come on the release liner 13. In another embodiment, the label 10 may be linerless. The support liner 13 may have a surface coating (a.k.a., release coating) or any low adherence surface or feature, upon which the adhesive layer 12 is laid, to facilitate the separation of the facestock 11 and adhesive layer 12 from the support liner 13.

The adhesive in the adhesive layer 12 may be any type of adhesive including pressure-sensitive adhesives, and non-limitative examples include a water-based acrylic, an emulsion adhesive, a hot melt including a UV hot melt, a rubber-based adhesive, a latex-based adhesive, a solvent-based adhesive, a silicon-based adhesive, a UV-curable adhesive, a LED-curable adhesive including a LED-UV-curable adhesive, a cross-linked adhesive, heat activated adhesive, adhesives for cold-stamping or hot-stamping, any combination thereof, etc. Similarly, in a variant, the adhesive can be permanent, while in another scenario, the adhesive can be removable and/or repositionable, allowing repositioning of the label 10 on the surface to be labelled. The adhesive of the adhesive layer 12 can also be a glove-friendly removable adhesive, for example. It is noted that any pressure-sensitive adhesive could be used. In some implementations, the adhesive of the adhesive layer 12 can be weakened or even neutralized at one or more locations using any adhesive neutralizing agent such as a varnish, an ink or UV varnish, a UV ink or the like. In some implementations, the adhesive 12 can be applied according to a certain pattern covering only a portion of the facestock 11 such that a remaining portion of the facestock undersurface is devoid of any adhesive. For example, the neck portion 10B could be without adhesive.

The adhesive layer 12 may have a thickness equal to or less than about 4.0 mil. More specifically, the adhesive layer 12 could have a thickness of between about 0.05 mil and 2.0 mil, and even more specifically, the adhesive layer 12 could have a thickness of between about 0.4 mil and 1.5 mil, or of between about 0.6 mil and 1.2 mil, or still more specifically, of between about 0.7 mil and 1.0 mil.

The release liner 13 may be paper based, or polymer based, with contemplated polymers including a polyester (PET), polypropylene, bi-axially oriented polypropylene (BOPP) or any other type of a polymer. For example, the release liner 13 may be a silicone or fluorosilicone coated support on which the adhesive layer 12 is harboured or retained, though other materials can be used, including waxes or other adhesive release coatings, etc, on a substrate. For example, if the support liner 13 is paper based, a low friction coating (e.g., silicone, fluorosilicone, or non-silicone based release coating) may be present to facilitate the peeling off of the facestock 11 and adhesive layer 12 from the support liner 13. Accordingly, when the release liner 13 is removed, the facestock 11 may be adhered to the vial surface, by the adhesive layer 12. In another embodiment, the release liner 13 may have imaging properties meaning that the manual writing with a pen or a pencil or with any type of impact instrument or impact printer can create a copy of the image on the release liner 13. In this case, when the facestock 11 is removed from the liner 13, the copy of the printed information or the image remains on the release liner 13 similar to carbon-copying. In another embodiment, the label 10 has a paper based facestock and a paper-based release liner 13. In another embodiment, the label 10 has a paper based facestock and a polymeric release liner 13. In another embodiment, the label 10 has a polymeric facestock 11 and a paper-based release liner 13. In another embodiment, the label 10 has a polymeric facestock 11 and a polymeric release liner 13. In another embodiment, the polymeric facestock 11 has an elongation value over 40% in either Machine Direction (MD) or Traverse Direction (TD) prior to breaking as measured according to ASTM D-882A (American Standard Testing Method). In another embodiment, the polymeric facestock 11 has an elongation value over 80% in either Machine Direction (MD) or Traverse Direction (TD) prior to breaking as measured according to ASTM D-882A.

In an embodiment, as shown in FIG. 3B the label may have additional layers such as an additional adhesive layer 14 and an additional support liner 15, in what may be known as a piggy-back label configuration. In the piggy-back configuration, the facestock 11, adhesive layer 12 and support liner 13 may be concurrently separated from the adhesive layer 14 and support liner 15.

Referring to FIG. 4, a plurality of the labels 10 are shown on a common release liner 13, the release liner 13 being in the form of a roll 20. In an embodiment, the facestock 11 covers all of the release liner 13 in the roll 20, with the labels 10 being die-cut so as to be removable from the release liner 13. In such a case, a waste portion of facestock 11, defining contours of the labels 10, would be present. As other embodiments, the release liner 13 may be in the form of a sheet (such as a letter size 8.5″×11″ or A4, or other sheet format) with rows and columns of the labels 10, or in the form of flat strips with rows of the labels 10, booklet, fanfold, etc. In an embodiment, when the labels 10 are intended for printing in a laser printer, a LED printer or any other type of printer in a sheet format, the release liner 13 may be resistant to heat without curling when it is passed through such a printer. Such release liners are also known as “layflat” liners. In some cases the layflat liners are thicker than regular release liners, and may be for example up to 7.0 mil in thickness. In an embodiment, the label 10 comes in a kit. The kit may include one or more of the labels 10, such as labels 10 on the release liner 13, and one or more matrix tubes 1, including or not the cap 2. In the kit, labels 10 may be pre-applied to the matrix tubes 1, or provided detached. The kit may include the presence of a cap(s) 2, already on the tube(s) 1, or not. The kit may also include the presence of a liquid or other substance in the tube 1. Some or all of the components of the kit may be sterile (i.e., sterilized) as an option.

Referring to FIG. 5, the plurality of labels 10 in the roll 20 are shown with the liner 13 having a main liner portion 13A and a plurality of removable liner portions 13B, with each label 10 overlapping a corresponding removable liner portion 13B, and the main liner portion 13A. The removable liner portions 13B can be positioned adjacent each other along a liner side edge 13F. A liner cut line may include notches 13D extending from the liner side edge 13F and creating gaps between adjacent removable liner portions 13B, or may include slits 13D′ (FIG. 7) instead of notches 13D such that the adjacent removable liner portions 13B are contiguous to each other. The main liner portion 13A may be separated from the removable liner portions 13B by a slit 13E, such that the main liner portion 13A is not directly attached to the removable liner portions 13B, the label 10 holding the liner portions 13A and 13B together. The slit 13E may be a continuous line, for instance die-cut in the liner 13. The slit 13E may be parallel to the liner side edges 13C and 13F, and may have other shapes.

In an embodiment, as an alternative to a continuous slit 13E, webs of about 1 to 2 mm may be left to disrupt the slit 13E. In other words, the removable liner portions 13B would be connected to the main liner portion 13A by one or more small connecting webs of liner. In this implementation, the user would break these small connecting liner portions to separate the removable liner portion 13B from the main liner portion 13A.

As observed, the labels 10 may be spaced from the liner side edges 13C and 13F, so as to facilitate the peeling of the label 10 from the removable liner portions 13B or the main liner portion 13A, respectively. For example, each label 10 may be spaced from the first liner side edge 13F by a distance of between about 1 mm and 30 mm, or more specifically by a distance of between about 1 mm and 5 mm. Alternatively, the labels 10 could extend all the way to the first liner side edge 13C and/or 13F and thereby be located adjacent the liner side edge 13C and/or 13F such that they cover entirely the removable liner portion 13B and/or the main liner portion 13A.

In an embodiment in which notches 13D are provided in the liner side edge 13F between adjacent removable liner portions 13B, the notches 13D may provide indications relative to the position of the labels 10 on the roll 20, in a sheet or in any other format, to a sensor used for printing an inscription, such as indicia, markings, instructions, graphics, logos, serial numbering, text, an image, a barcode or the like, and/or for encoding a wireless tag such as RFID, NFC and the like on the labels, such as a sensor of a label printer for example, thereby facilitating the printing of an inscription on the labels 10. It is also possible to add label position indicators 13G on the liner 13 or on the facestock 11, for position detection by a sensor associated with a printer. The label position indicators 13G may be darker lines or marks, visually detectable features, at periodic locations along the roll 20 or on a liner sheet.

Still referring to FIG. 5, tear lines 13H may be provided to separate the liner 13 in segments. For example, the tear lines 13H may be formed in the liner 13 between each label 10, such that a label 10 and its associated portion of liner 13 may be detached from the roll 20, or from a sheet, booklet, fanfold or any other format of label 10 and liner 13. When the label 10 is detached with its associated portion of liner 13, it may come with both the removable liner portion 13B and the main liner portion 13A. In an embodiment, if facestock waste portion is not removed from around the labels 10, then a facecut slit may be present in the facestock 11 along or parallel (e.g., generally parallel) to the tear lines 13H and/or along or parallel (e.g., generally parallel) to the slit 13E, to facilitate the release of the labels 10 from the surrounding matrix of waste material when the removable portion is lifted.

The facestock in the roll 20 can also include a waste portion affixed to the label receiving surface of the liner 13 and surrounding entirely or partially the labels 10, or alternatively, the facestock may not include a waste portion and may only include the labels 10 when the roll 20 is provided to a user.

The removable liner portions 13B, which can be separated from the main liner portion 13A and which remain affixed to the label 10 when the label 10 is peeled off the liner 13, may facilitate the peeling of the labels 10 off the liner 13. The removable liner portions 13B may also facilitate the manipulation of the label 10 once the label 10 has been removed from the liner 13, and the affixing of the label 10 to the desired surface. For example, the removable liner portion 13B can prevent the label 10 from sticking to the user's finger or gloves, which would impede the manipulation of the label 10. Moreover, by eliminating the need for the user to directly touch the label's adhesive, this configuration can reduce or eliminate the risk for a user to have an allergic reaction upon contact with the adhesive to natural rubber or latex or other ingredients which may be used in the adhesive. The removable liner portion 13B may also act as a reinforcement to rigidify or increase the stiffness of label 10 to reduce the bending of the label 10 during the peeling, handling and affixing of the label 10.

Once the label 10 has been peeled off from the main liner portion 13A, the label 10 may have different physical and/or mechanical properties at different areas of the label 10. Specifically, since the removable liner portion 13B is still affixed to a portion of the label, that portion of the label together with the removable liner portion 13B has a different stiffness, and more specifically a greater stiffness, i.e. a greater Young's modulus, compared to the label portion without the main liner portion 13A thereon.

Also due to the removable liner portion 13B remaining affixed to a portion of the label 10, the portions of the label 10 with and without the liner 13 may also have different thickness, tensile behaviour, optical transparency, bending stiffness and/or rotational stiffness. This can facilitate the manipulation of the label 10 which may otherwise be relatively flimsy and may be difficult to direct to the tube 1 on which the label 10 is to be affixed.

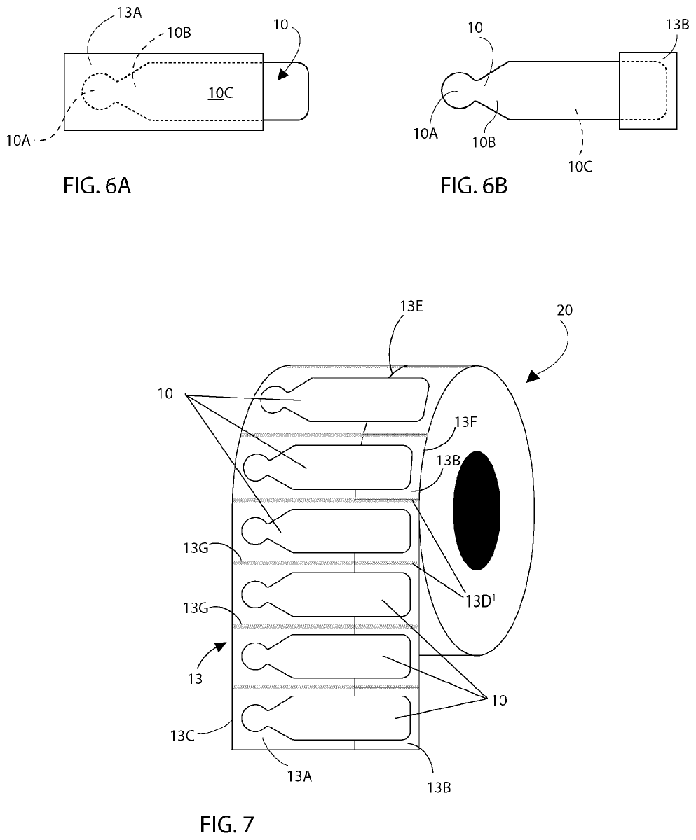

FIG. 6A shows an embodiment differing to that of the roll 20 of FIG. 5, in that the removable liner portion 13A covers the end label portion 10A, the neck 10B and a portion of the main label portion 10C, while an end of the main label portion 10C would have its adhesive surface exposed by the peeling of the label 10 from the liner portion 13B. It is however contemplated to have the removable liner portion 13A cover only the end label portion 10A, or the end label portion 10A and only part of the neck 10B.

Therefore, in such an embodiment, the user would adhere the main label portion 10C to the side surface of the tube 1 (and possibly to the cap 2), to then peel the removable liner portion 13A off and adhere the end label portion 10A to the bottom face 1A.

FIG. 6B shows an embodiment that differs from that of FIG. 6A and corresponding to the embodiment of FIG. 5, in that the liner portion 13B covering the main label portion 10C is the removable liner portion that remains adhered to the label 10, while the end label portion 10A and the neck 10B have their adhesive surface exposed by the peeling of the label 10 from the main liner portion 13A (FIG. 5). It is also possible to have the liner portion 13A cover only the end label portion 10A, or the end label portion 10A and only part of the neck 10B.

In such an embodiment, the user would first adhere the end label portion 10A to the bottom face 1A, to then peel the removable liner portion 13B off and adhere the the main label portion 10C to the side surface of the tube 1 (and possibly to the cap 2). Due to the relatively small size of the bottom face 1A, may be easier to adhere the end label portion 10A to the bottom face 1A, to then adhere the main label portion 10C to the lateral surface of the tube 1, the latter operation being relatively easier due to the larger area of the lateral surface of the tube 1 in comparison to its bottom face 1A. As observed in FIG. 6B, the removable liner portion 13B may only cover an end of the main label portion 10C, of sufficiently large dimension to allow finger manipulation of the label 10 via the removable liner portion 13B. The removable liner portion 13B may extend longer in direction LC, and may potentially extend to a part of the neck portion 10B of the label 10. However, in an embodiment, as the neck portion 10B of the label 10 may be deformed during use, the absence of the removable liner portion 13B on the neck portion 10B may facilitate the manipulations and the compliance of the label 10.

Referring to FIG. 7, an alternative embodiment to that of the roll 20 is shown. Instead of notches 13D (FIG. 5) between the removable liner portions 13B, slits 13D′ may be provided, to cause a separation between the removable liner portions 13A. The slits 13D′ may be die-cut into the liner 13, and may also extend into the waste portions of facestock 11 if present. The roll 20 of FIG. 7, may or may not have some of the features of the roll 20 of FIG. 5, such as pre-printed data, writable or printable zones, webs of liner between removable liner portions 13B and main liner portion 13A, label position indicators 13G, the tear lines 13H. Likewise, the labels 10 in the roll 20 of FIG. 7 may be as described in any of the embodiments described herein.

As a possibility, the removable liner portion 13B and/or the main liner portion 13A or part thereof may not be removed and may be left affixed to the label 10, while the main label portion 10C or part of it is adhered to the tube 1. The removable liner portion 13B may remain attached to the tube 1 via the label 10. In these implementations, the removable liner portion 13B may therefore be used as a tag to provide information to the user, such as for example information regarding the tube 1 or the content of the tube 1. As a possibility, an inscription could be printed on the removable liner portion 13B. The removable liner portion 13B could also be blank to allow the user to manually write an indication thereon, or to receive another label containing an indication. The removable liner portion 13B could also be used to remove the label 10 from the tube 1 if desired. The user could simply grasp the removable liner portion 13B and move it towards a remainder of the label 10 to peel the label 10 off from the tube 1. In some implementations, the label 10 may incorporate one or more tear lines (e.g., 10C′), perforations or cuts which would allow the user to tear away a portion of the label 10 prior, during or after the application of the label 10 or could allow the label to be used for tamper-evident applications, for example. Other features associated with the removable liner portion 13B and main liner portion 13A are as described in U.S. patent application Ser. No. 17/092,719, filed on Nov. 9, 2020 and incorporated herein by reference.

A method for applying the label 10 onto one of the matrix tubes 1 may be defined as including one or more of: pressing the end label portion 10A of the label 10 against a bottom face 1A of a matrix tube 1; deforming the label 1 at the neck portion 10B (e.g., folding, pivoting, bending, rotating) to move the main label portion 10C toward a side surface 1C of the matrix tube 1; and pressing the main label portion 10C of the label 10 against the side surface 1C of the matrix tube 1. The method may include other steps such as: inscribing or printing data on the label 10; removing the label 10 from a release liner 13; applying the labels manually to the matrix tube 1; scanning a barcode on the label; and/or retrieving an identify of the matrix tube using the scanning of the barcode. It is contemplated a use of a label applicator or a robotic device for applying labels 10 to the matrix tube 1, or using a print-and-apply or a peel-and-apply stand alone device or an integrated device as a part of robotic or automation system for applying labels 10 to the matrix tube 1. Printing on the label 10 already on the tube 1 is possible, using a tube printer device.

Stated differently, the method for applying a label such as 10 onto a matrix tube 1, may include: pressing an end label portion of a label against a bottom face of a matrix tube; deforming the label at neck portion to move a main label portion toward a side surface of the matrix tube; and pressing the main label portion of the label against the side surface of the matrix tube. There may be performed removing a first liner portion from the end label portion and keeping a second liner portion on the main label portion prior to pressing the end label portion against the bottom face of the matrix tube, and removing the second liner portion from the main label portion prior to pressing the main label portion against the side surface of the matrix tube. There may be performed inscribing or printing data on the label. There may be included scanning a barcode on the label. There may be included retrieving an identity of the matrix tube or its content using the scanning of the barcode. There may be included storing data or an identity of the matrix tube or its content using the scanning of the barcode.

In addition to the embodiments shown herein, the label 10 can be positioned in any orientation on a roll or sheet, such as landscape, portrait, vertical, with any appropriate angle. An orientation can be provided to facilitate automatic peeling from a peeling device or from a robotic device.

The label configurations and methods described hereinafter may be used in any industry where labels are used. Some implementations of the label configurations and methods may further be particularly well suited for use in certain industries. In particular, the labels used in some industries—such as in biotechnology, biomedicine, cell banking, tissue banking and other fields involving cryogenic bio-preservation or freezing of biological specimen—are typically required to be thinner and more flexible than in other fields and are therefore substantially more difficult to peel using prior art methods. For example, it has been shown that cryogenic label materials that are intended for use at −80 C and below such as inside liquid nitrogen tanks at −196 C or below or that are exposed to liquid helium at −269 C may have facestock elongation values between 10% and 500% or even more. It will however be understood that the label configurations and methods described hereinafter are not limited to use in these fields, and may be used instead in other fields such as packaging, automotive, electrical, electronics, avionics, aerospace, food, chemical, agricultural, fashion, gas and oil, plumbing, heavy industrial, light industrial, construction, or any other suitable filed or industry.

1. A label for matrix tube comprising:
a facestock configured to have data on a first surface thereof, and
an adhesive layer on a second surface of the facestock,
wherein the facestock defines an end label portion, a main label portion and a neck portion between the end label portion and the main label portion, the neck portion being narrower than the end label portion and the main label portion.

2. The label according to claim 1, wherein the end label portion has a generally circular shape.

3. The label according to claim 2, wherein the end label portion has a diameter of at most 6.5 mm.

4. The label according to claim 1, wherein the neck portion is narrower than the end label portion by having a minimum width being between 10% to 80% of a width of the end label portion.

5. The label according to claim 4, wherein the neck portion has a constant width from the end label portion to the main label portion.

6. The label according to claim 4, wherein the neck portion flares from the end label portion to the main label portion.

7. The label according to claim 1, wherein the main label portion has a generally rectangular or square shape.

8. (canceled)

9. The label according to claim 1, wherein the main label portion has a maximum width a ranging from 3.0 mm to 40.0 mm.

10. The label according to claim 9, wherein the main label portion is wider than the end label portion.

11. The label according to claim 1, wherein the end label portion, the neck portion and the main label portion extend in a lengthwise of the label, the end label portion having a length ranging between 3.0 to 6.5 mm.

12. The label according to claim 11, wherein the neck portion has a length ranging between 1.0 mm and 15.0 mm.

13. The label according to claim 11, wherein the main label portion has a length of at least 5.0 mm.

14. The label according to claim 1, wherein the main label portion has a tear line transverse to a length thereof.

15. The label according to claim 1, wherein the end label portion and/or the main label portion has a barcode, data and/or ink thereon.

16.-17. (canceled)

18. The label according to claim 1, further including a wireless communication tag or related component in the label.

19. The label according to claim 1, further including a support liner, the adhesive layer being between the facestock and the support liner, for releasable connection of the facestock to the support liner.

20. The label according to claim 19, wherein the support liner is in adhered to another support liner by another layer of adhesive in a piggy-back configuration.

21. The label according to claim 19, wherein the support liner has a first liner portion and a second liner portion separated by at least one slit.

22.-27. (canceled)

28. A kit comprising:
at least one of the label according to claim 1, and
a matrix tube.

29. A method for applying a label onto a matrix tube, comprising:
pressing an end label portion of a label against a bottom face of a matrix tube;
deforming the label at neck portion to move a main label portion toward a side surface of the matrix tube; and
pressing the main label portion of the label against the side surface of the matrix tube.

30.-34. (canceled)
